(19) United States
(12) Patent Application Publication
(10) Pub. No.: US 2025/0229041 A1
(43) Pub. Date: Jul. 17, 2025

(54) Oil Diffuser Device

(71) Applicant: Kenneth Gibbons, Mount Pearl (CA)

(72) Inventor: Kenneth Gibbons, Mount Pearl (CA)

(21) Appl. No.: 18/897,642

(22) Filed: Sep. 26, 2024

Related U.S. Application Data
(60) Provisional application No. 63/621,692, filed on Jan. 17, 2024.

(51) Int. Cl. A61M 11/04; A61M 21/00; A61M 21/02

(52) U.S. Cl. CPC A61M 11/042; A61M 21/02; A61M 2021/0044; A61M 2021/0077; A61M 2205/502; A61M 2205/8262

(57) ABSTRACT

An oil diffuser device that is a plug-in heat and fan device for diffusing CBD oil into the air. The oil diffuser device comprises a body component with a plurality of ventilation holes for retaining an amount of CBD oil or other suitable oil. A plug-in component comprises a heat source that draws power from an electrical outlet. The plug-in component also comprises a fan for dispersing the heated oil vapors through the plurality of ventilation holes. Users simply attach the body component with CBD oil to the plug-in component and then insert it into an electrical outlet to heat the CBD oil and release the heated oil vapors into the air. Overall, the device can be used to relax people who suffer from anxiety, depression, or stress.

### CROSS-REFERENCE TO RELATED APPLICATION

The present application claims priority to, and the benefit of, U.S. Provisional Application No. 63/621,692, which was filed on Jan. 17, 2024, and is incorporated herein by reference in its entirety.

### FIELD OF THE INVENTION

The present invention relates generally to the field of oil diffuser devices. More specifically, the present invention relates to an electrical outlet plug-in device that diffuses CBD oil and other similar products. Accordingly, the present disclosure makes specific reference thereto. Nonetheless, it is to be appreciated that aspects of the present invention are also equally applicable to other like applications, devices and methods of manufacture.

### BACKGROUND

By way of background, this invention relates to improvements in oil diffuser devices. Generally, people suffering from anxiety, depression or stress may be looking for new and easier ways to relax. Different types of oils and vapors may alleviate stress and anxiety for some people. Further, people may get frustrated applying CBD oil and other similar products to their skin.

Cannabidiol oil, or CBD oil derived from cannabis, is a popular natural remedy for many ailments. CBD oil benefits include pain relief and relaxation without any mind-altering effects. However, there is a need for an aroma diffuser that is able to diffuse the aroma to the surroundings without changing the therapeutic and chemical properties of the CBD oil and to provide consistent flow of aroma at a controllable rate. It is also desirable that the aroma diffuser can be readily and easily serviceable. The present invention was developed in consideration of these needs.

Accordingly, there is a demand for an improved oil diffuser device that provides users with an electrical outlet plug-in device that diffuses CBD oil and other similar products. More particularly, there is a demand for an oil diffuser device that offers a way for people to relax when suffering from anxiety or stress.

Therefore, there exists a long felt need in the art for an oil diffuser device that provides users with an electrical outlet plug-in device that diffuses CBD oil and other similar products. There is also a long felt need in the art for an oil diffuser device that features ventilation holes that release vapor when heated via a fan. Further, there is a long felt need in the art for an oil diffuser device that enables users to choose from different concentrations of oils to release them into the air in a confined space. Moreover, there is a long felt need in the art for a device that offers a way for people to relax when suffering from anxiety or stress. Further, there is a long felt need in the art for an oil diffuser device that is used to relax people who suffer from anxiety, depression or stress. Finally, there is a long felt need in the art for an oil diffuser device that draws power from an electrical outlet.

The subject matter disclosed and claimed herein, in one embodiment thereof, comprises an oil diffuser device. The device is a plug-in heat and fan device for diffusing CBD oil into the air. The oil diffuser device comprises a body component that is configured to retain an amount of CBD oil or other suitable oil. The body component comprises a plurality of ventilation holes for dispersing the heated oil vapors. A plug-in component comprises a heat source that draws power from an electrical outlet. The plug-in component also comprises a fan for dispersing the heated oil vapors through the plurality of ventilation holes. Users simply attach the body component with CBD oil to the plug-in component and then insert it into an electrical outlet to heat the CBD oil and release the heated oil vapors into the air. Further, users can control the concentration levels of the CBD oil being dispersed, while the device is plugged in. Overall, the device can be used to relax people who suffer from anxiety, depression, or stress.

In this manner, the oil diffuser device of the present invention accomplishes all of the forgoing objectives and provides users with a device that provides users with an electrical outlet plug that diffuses CBD oil. The device is a plug-in device that releases vapor when heated via a fan. The device can utilize different oils and concentrations of oils.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an oil diffuser device. The device is a plug-in heat and fan device for diffusing CBD oil into the air. The oil diffuser device comprises a body component that is configured to retain an amount of CBD oil or other suitable oil. The body component comprises a plurality of ventilation holes for dispersing the heated oil vapors. A plug-in component comprises a heat source that draws power from an electrical outlet. The plug-in component also comprises a fan for dispersing the heated oil vapors through the plurality of ventilation holes. Users simply attach the body component with the CBD oil to the plug-in component and then insert it into an electrical outlet to heat the CBD oil and release the heated oil vapors into the air. Further, users can control the concentration levels of the CBD oil being dispersed. Overall, the device can be used to relax people who suffer from anxiety, depression, or stress.

In one embodiment, the oil diffuser device is an oil diffuser type air freshener for providing a desired oil, fragrance, or scent to the surrounding environment. In addition, the air freshener or the oil diffuser is a plug-in type that is plugged into a standard, residential electrical outlet that provides both electrical power and physical support for the device. Specifically, the oil diffuser device diffuses CBD oil, or any other suitable oil. The CBD oil is heated via a heat source and diffused as a vapor through a plurality of ventilation holes.

In one embodiment, the oil diffuser device comprises a body component and a plug-in component. The body component is configured to retain an amount of CBD oil or other suitable oil. The body component comprises a bottom surface and a curved sidewall configured in a cylindrical shape defining a cavity, with an open top that allows access to the cavity. A user can then fill the body component with an amount of CBD oil. Further, the open top comprises a plurality of external threads which mate with a plurality of internal threads on the plug-in component. Thus, the body component can be screwed into the plug-in component, during use.

In one embodiment, the body component is configured as a semi cylindrical shape which may be made of glass, ceramic materials or metal-based materials to retain the CBD oils or other suitable oils. Further, the open top area comprises a plurality of ventilation holes, where the CBD vapors are then released when the CBD oils are heated and pressurized. Specifically, the body component comprises a plurality of ventilation holes which permit the airflow flowing from the fan and the heated oil vapors generated in the body component to be combined and exit the device. These vapors are directed upwards by the airflow from a fan and passed through the plurality of ventilation holes on the open top to the surrounding atmosphere. Thus, CBD oil is atomized, ejected and sprayed out of the body component via the fan and the plurality of ventilation holes.

In one embodiment, the plug-in component comprises a power plug component for securing the device to a wall outlet or other power source. Specifically, the power plug component is a Universal Series Bus (USB) plug, a car charger connector, or an ordinary electrical plug. In use, the power plug component is inserted into the insertion slot of a power socket. Thus, a power plug component extends from the plug-in component. In one embodiment, the power plug component can also be folded up into the plug-in component for storage. Accordingly, the power plug component can be a standard, residential electrical plug with prongs that can be plugged into a standard, residential, wall electrical outlet. Thus, the device can be secured to the wall electrical outlet and suspended on the wall.

In one embodiment, the plug-in component comprises a fan. The fan is mounted on the plug-in component to direct air and CBD oil vapors from the body component out through the plurality of ventilation holes of the device. The fan can be any suitable fan as is known in the art. Further, the fan can be any suitable size and shape as is known in the art, depending on the size of the plug-in component where it is housed. Further, the fan is typically activated by plugging in the power plug component to an electrical wall outlet or other suitable power device.

In one embodiment, the plug-in component comprises a heat source that causes the CBD oil to stir into bubbles before breaking into smaller molecules. The heating has the effect of breaking down the molecules of the CBD oil. This heat source includes a wire wound resistor and a thermostat which is embedded into the plug-in component, along with the heat source. The heat source can be any suitable heat source as is known in the art. Further, the heat source can be any suitable shape and size as is known in the art, depending on the size of the plug-in component where it is housed. The heat source provides low heating below a temperature of 5O° C., preferably below a temperature of 35° C., depending on the chemical properties of the CBD oil used. Heated oil vapors are released when the CBD oils are heated and pressurized. These vapors are directed upwards by the airflow from the fan and the heated oil vapors are passed through the ventilated holes of the top of the body component.

In one embodiment, a light source can be disposed in the plug-in component and electrically coupled to the power plug component via the control electronics. The light source can include one or more LEDs. The light from the light source can provide an aesthetically pleasing appearance and can enhance the therapeutic operation of the device along with the aroma. A light control button can control color and/or brightness of the light source.

In one embodiment, the device has a faceplate coupled to the body component. The faceplate can have a width or diameter greater than a width or diameter of the body component in order to cover and hide the body component when viewed straight on. In addition, the faceplate can be non-plastic. In one aspect, the faceplate can be or can comprise ceramic that is fire hardened and glazed. The faceplate can also cover the body component for a more aesthetically pleasing appearance.

In one embodiment, the body components can retain different amounts and different concentrations of CBD oil. Thus, the device enables users to choose from different concentrations of CBD oils to release the oils into the air in a confined space. Further, users can control the concentration level of the CBD oil being dispersed.

In one embodiment, the device can be scaled to a size to suit the application of the user. Thus, the oil diffuser device offers the flexibility of scaling the size of the device depending on the application needs by varying the dimensions of the components accordingly.

In yet another embodiment, the oil diffuser device comprises a plurality of indicia.

In yet another embodiment, a method of diffusing CBD oil into the air is disclosed. The method includes the steps of providing an oil diffuser device comprising a body component with ventilation holes and a plug-in component. The method also comprises inserting CBD oil into the body component. Further, the method comprises securing the body component to the plug-in component. The method also comprises inserting the plug-in component into an electrical outlet. The method comprises heating the CBD oil with a heat source in the plug-in component. Finally, the method comprises dispersing heated CBD oil vapors through the plurality of ventilation holes via a fan.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the oil diffuser device of the present invention showing the device with different oils in accordance with the disclosed architecture;

FIG. 2 illustrates a perspective view of one embodiment of the oil diffuser device of the present invention showing different oil concentrations available in accordance with the disclosed architecture;

FIG. 3 illustrates a perspective view of one embodiment of the oil diffuser device of the present invention showing how the oil is attached to the plug-in component in accordance with the disclosed architecture;

FIG. 4 illustrates a perspective view of one embodiment of the oil diffuser device of the present invention showing how the device is inserted into an electrical outlet in accordance with the disclosed architecture;

FIG. 5 illustrates a perspective view of one embodiment of the oil diffuser device of the present invention in use in accordance with the disclosed architecture; and

FIG. 6 illustrates a flowchart showing the method of diffusing CBD oil into the air in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for an oil diffuser device that provides users with an electrical outlet plug-in device that diffuses CBD oil and other similar products. There is also a long felt need in the art for an oil diffuser device that features ventilation holes that release vapor when heated via a fan. Further, there is a long felt need in the art for an oil diffuser device that enables users to choose from different concentrations of oils to release them into the air in a confined space. Moreover, there is a long felt need in the art for a device that offers a way for people to relax when suffering from anxiety or stress. Further, there is a long felt need in the art for an oil diffuser device that is used to relax people who suffer from anxiety, depression or stress. Finally, there is a long felt need in the art for an oil diffuser device that draws power from an electrical outlet.

The present invention, in one exemplary embodiment, is a novel oil diffuser device. The oil diffuser device comprises a body component that is configured to retain an amount of CBD oil or other suitable oil and a plurality of ventilation holes for dispersing the heated oil vapors. A plug-in component comprises a heat source that draws power from an electrical outlet. The plug-in component also comprises a fan for dispersing the heated oil vapors through the plurality of ventilation holes. Users simply attach the body component with CBD oil to the plug-in component and then insert it into an electrical outlet to heat the CBD oil and release the heated oil vapors into the air. Overall, the device can be used to relax people who suffer from anxiety, depression, or stress. The present invention also includes a novel method of diffusing CBD oil into the air. The method includes the steps of providing an oil diffuser device comprising a body component with ventilation holes and a plug-in component. The method also comprises inserting CBD oil into the body component. Further, the method comprises securing the body component to the plug-in component. The method also comprises inserting the plug-in component into an electrical outlet. The method comprises heating the CBD oil with a heat source in the plug-in component. Finally, the method comprises dispersing heated CBD oil vapors through the plurality of ventilation holes via a fan.

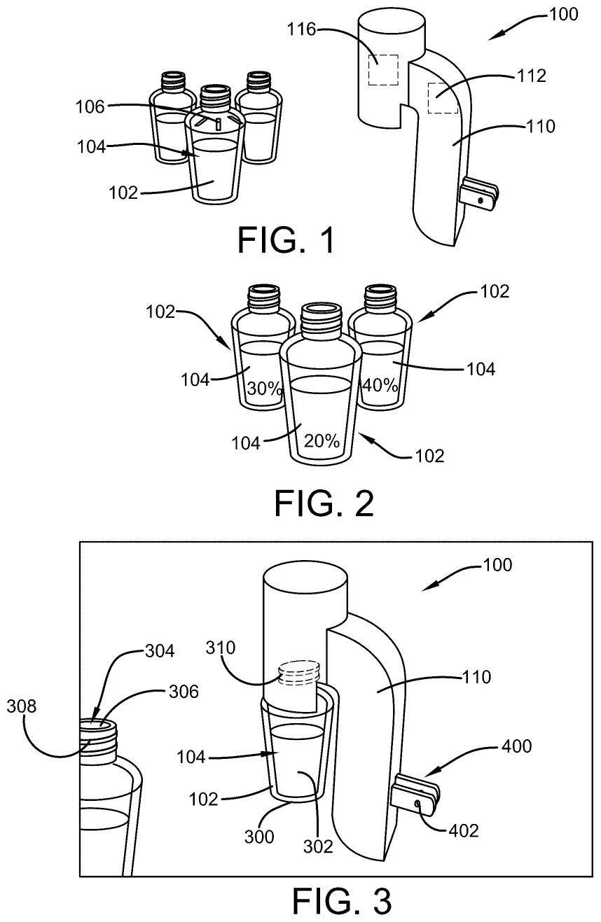

Referring initially to the drawings, FIG. 1 illustrates a perspective view of one embodiment of the oil diffuser device 100 of the present invention. In the present embodiment, the oil diffuser device 100 is an improved oil diffuser device 100 that provides a user with a plug-in heat and fan device for diffusing CBD oil into the air. Specifically, the oil diffuser device 100 comprises a body component 102 that is configured to retain an amount of CBD oil 104 or other suitable oil. The body component 102 comprises a plurality of ventilation holes 106 for dispersing the heated oil vapors 108. A plug-in component 110 comprises a heat source 112 that draws power from an electrical outlet 114. The plug-in component 110 also comprises a fan 116 for dispersing the heated oil vapors 108 through the plurality of ventilation holes 106. Users simply attach the body component 102 with the CBD oil 104 to the plug-in component 110 and then insert it into an electrical outlet 114 to heat the CBD oil 104 and release the heated oil vapors 108 into the air. Overall, the device 100 can be used to relax people who suffer from anxiety, depression, or stress.

Generally, the oil diffuser device 100 is an oil diffuser type air freshener for providing a desired oil, fragrance, or scent to the surrounding environment. In addition, the air freshener or the oil diffuser 100 is a plug-in type that is plugged into a standard, residential electrical outlet 114 that provides both electrical power and physical support for the device 100. Specifically, the oil diffuser device 100 diffuses CBD oil 104, or any other suitable oil. The CBD oil 104 is heated via a heat source 112 and diffused as a vapor 108 through a plurality of ventilation holes 106.

As shown in FIG. 2, the body components 102 can retain different amounts and different concentrations of CBD oil 104. Thus, the device 100 enables users to choose from different concentrations of CBD oils 104 to release the oils into the air in a confined space. Further, users can control the concentration level of the CBD oil 104 being dispersed. Additionally, any suitable type of CBD oil 104 can be utilized as is known in the art, depending on the needs and/or wants of a user. Further, any suitable concentration of CBD oil 104 can be utilized with the device 100, as is known in the art depending on the needs and/or wants of a user.

As shown in FIG. 3, the oil diffuser device 100 comprises a body component 102 and a plug-in component 110. The body component 102 is configured to retain an amount of CBD oil 104 or other suitable oil. The body component 102 comprises a bottom surface 300 and a curved sidewall 302 configured in a cylindrical shape defining a cavity 304, with an open top 306 that allows access to the cavity 304. A user can then fill the body component 102 with an amount of CBD oil 104. Further, the open top 306 comprises a plurality of external threads 308 which mate with a plurality of internal threads 310 on the plug-in component 110. Thus, the body component 102 can be screwed into the plug-in component 110, during use. In another embodiment, the body component 102 is merely press-fit or snap-fit into the plug-in component 110 for use.

Further, the body component 102 is configured as a semi cylindrical shape which may be made of glass, ceramic materials or metal-based materials to retain the CBD oils 104 or other suitable oils. Further, the open top area 306 comprises a plurality of ventilation holes 106, where the CBD vapors 108 are then released when the CBD oils 104 are heated and pressurized. Specifically, the body component 102 comprises a plurality of ventilation holes 106 which permit the airflow flowing from the fan 116 and the heated oil vapors 108 generated in the body component 102 to be combined and exit the device 100. These vapors 108 are directed upwards by the airflow from the fan 116 and passed through the plurality of ventilation holes 106 on the open top 306 to the surrounding atmosphere. Thus, the CBD oil 104 is atomized, ejected and sprayed out of the body component 102 via the fan 116 and the plurality of ventilation holes 106. The plurality of ventilation holes 106 are typically positioned near the open top 306 of the body component 102 but can be positioned in any suitable position, as is known in the art. Any suitable number of ventilation holes 106 can be utilized on the body component 102, depending on how much CBD oil vapors 108 a user wishes to release into the environment at one time. More vapors 108 can be released at one time, with more ventilation holes 106 and less vapors 108 can be released at one time, with less ventilation holes 106.

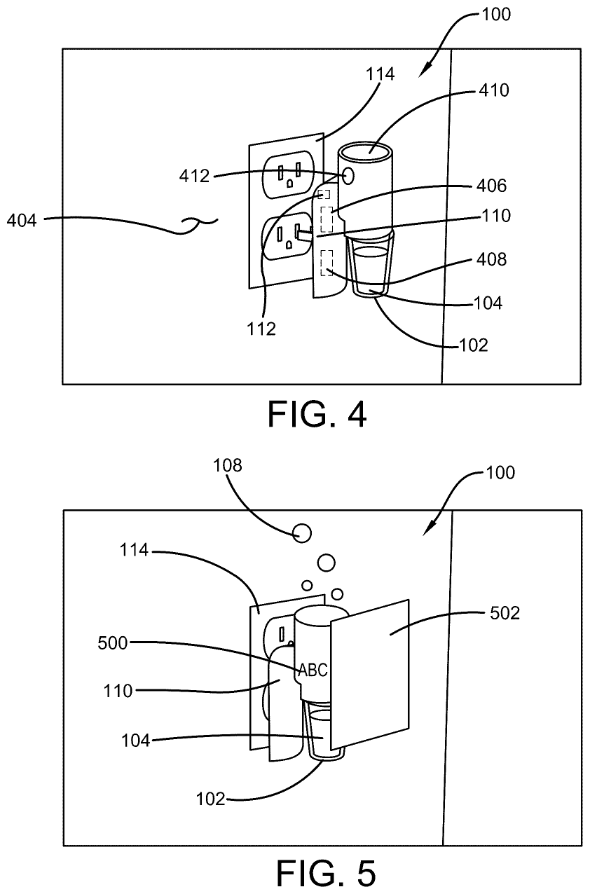

As shown in FIG. 4, the plug-in component 110 comprises a power plug component 400 for securing the device 100 to a wall outlet 114 or other power source. Specifically, the power plug component 400 is a Universal Series Bus (USB) plug, a car charger connector, or an ordinary electrical plug. In use, the power plug component 400 is inserted into the insertion slot of a power socket 114. Thus, a power plug component 400 extends from the plug-in component 110. In one embodiment, the power plug component 400 can also be folded up into the plug-in component 110 for storage. Accordingly, the power plug component 400 can be a standard, residential electrical plug with prongs 402 that can be plugged into a standard, residential, wall electrical outlet 114. Thus, the device 100 can be secured to the wall electrical outlet 114 and suspended on the wall 404.

Furthermore, the plug-in component 110 comprises a fan 116. The fan 116 is mounted on the plug-in component 110 to direct air and CBD oil vapors 108 from the body component 102 out through the plurality of ventilation holes 106 of the device 100. The fan 116 can be any suitable fan as is known in the art. Further, the fan 116 can be any suitable size and shape as is known in the art, depending on the size of the plug-in component 110 where it is housed. Further, the fan 116 is typically activated by plugging in the power plug component 400 to an electrical wall outlet 114 or other suitable power device.

Additionally, the plug-in component 110 comprises a heat source 112 that causes the CBD oil 104 to stir into bubbles before breaking into smaller molecules. The heating has the effect of breaking down the molecules of the CBD oil 104. This heat source 112 includes a wire wound resistor 406 and a thermostat 408 which is embedded into the plug-in component 110, along with the heat source 112. The heat source 112 can be any suitable heat source as is known in the art. Further, the heat source 112 can be any suitable shape and size as is known in the art, depending on the size of the plug-in component 110 where it is housed. The heat source 112 provides low heating below a temperature of 5O° C., preferably below a temperature of 35° C., depending on the chemical properties of the CBD oil 104 used. Heated oil vapors 108 are released when the CBD oils 104 are heated and pressurized. These vapors 108 are directed upwards by the airflow from the fan 116 and the heated oil vapors 108 are passed through the ventilated holes 106 of the top 306 of the body component 102.

Further, a light source 410 can be disposed in the plug-in component 110 and electrically coupled to the power plug component 400 via the control electronics. The light source 410 can include one or more LEDs. The light from the light source 410 can provide an aesthetically pleasing appearance and can enhance the therapeutic operation of the device 100 along with the aroma. A light control button 412 can control color and/or brightness of the light source 410, as well as can operate as a power button, manually turning the light source 410 off and on, as needed.

As shown in FIG. 5, in one embodiment, the device 100 has a faceplate 502 coupled to the body component 102. The faceplate 502 can have a width or diameter greater than a width or diameter of the body component 102 in order to cover and hide the body component 102 when viewed straight on. In addition, the faceplate 502 can be non-plastic. In one aspect, the faceplate 502 can be or can comprise ceramic that is fire hardened and glazed. The faceplate 502 can also cover the body component 102 for a more aesthetically pleasing appearance, and can comprise different colors and designs, depending on the needs and/or wants of a user.

In one embodiment, the device 100 can be scaled to a size to suit the application of the user. Thus, the oil diffuser device 100 offers the flexibility of scaling the size of the device 100 depending on the application needs by varying the dimensions of the components accordingly.

In yet another embodiment, the oil diffuser device 100 comprises a plurality of indicia 500. The body component 102 of the device 100 may include advertising, a trademark, or other letters, designs, or characters, printed, painted, stamped, or integrated into the body component 102, or any other indicia 500 as is known in the art. Specifically, any suitable indicia 500 as is known in the art can be included, such as but not limited to, patterns, logos, emblems, images, symbols, designs, letters, words, characters, animals, advertisements, brands, etc., that may or may not be CBD oil, diffuser, or brand related.

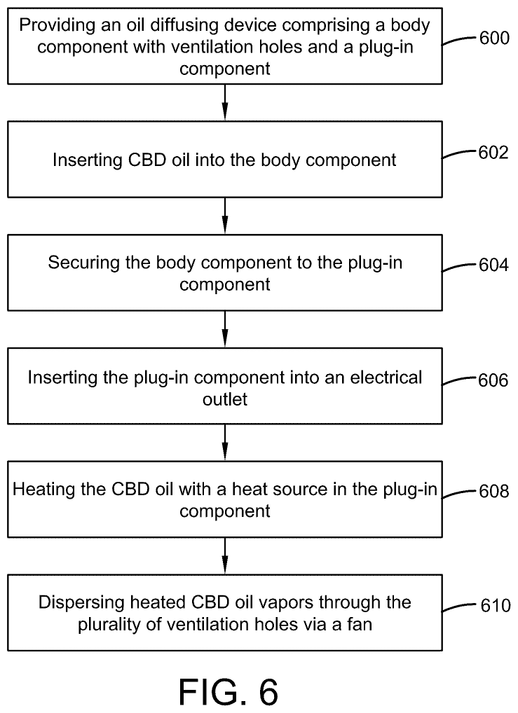

FIG. 6 illustrates a flowchart of the method of diffusing CBD oil into the air. The method includes the steps of at 600, providing an oil diffuser device comprising a body component with ventilation holes and a plug-in component. The method also comprises at 602, inserting CBD oil into the body component. Further, the method comprises at 604, securing the body component to the plug-in component. The method also comprises at 606, inserting the plug-in component into an electrical outlet. The method comprises at 608, heating the CBD oil with a heat source in the plug-in component. Finally, the method comprises at 610, dispersing heated CBD oil vapors through the plurality of ventilation holes via a fan.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “oil diffuser device”, “diffuser device”, and “device” are interchangeable and refer to the oil diffuser device 100 of the present invention.

Notwithstanding the forgoing, the oil diffuser device 100 of the present invention can be of any suitable size and configuration as is known in the art without affecting the overall concept of the invention, provided that it accomplishes the above stated objectives. One of ordinary skill in the art will appreciate that the oil diffuser device 100 as shown in FIGS. 1-6 is for illustrative purposes only, and that many other sizes and shapes of the oil diffuser device 100 are well within the scope of the present disclosure. Although the dimensions of the oil diffuser device 100 are important design parameters for user convenience, the oil diffuser device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An oil diffuser device that provides a user with a plug-in device for diffusing CBD oil into the air, the oil diffuser device comprising:
a body component; and
a plug-in component;
wherein the body component is configured to retain an amount of CBD oil;
wherein the body component with the CBD oil is secured to the plug-in component; and
further wherein the plug-in component is inserted into an electrical outlet for power to heat the CBD oil and release heated CBD oil vapors into the air.

2. The oil diffuser device of claim 1, wherein the body component retains different amounts and different concentrations of CBD oil.

3. The oil diffuser device of claim 2, wherein the body component comprises a bottom surface and a curved sidewall configured in a cylindrical shape defining a cavity, with an open top that allows access to the cavity.

4. The oil diffuser device of claim 3, wherein the open top comprises a plurality of external threads which mate with a plurality of internal threads on the plug-in component.

5. The oil diffuser device of claim 4, wherein the body component is configured as a semi cylindrical shape and made of glass, ceramic materials or metal-based materials to retain the CBD oil.

6. The oil diffuser device of claim 5, wherein the body component comprises a plurality of ventilation holes positioned near the open top.

7. The oil diffuser device of claim 6, wherein the plug-in component comprises a power plug component for securing to the electrical outlet.

8. The oil diffuser device of claim 7, wherein the power plug component is a Universal Series Bus (USB) plug, a car charger connector, or a conventional electrical plug.

9. The oil diffuser device of claim 8, wherein the power plug component is a standard, residential electrical plug with prongs that are plugged into a standard, residential, wall electrical outlet, to secure and suspend the oil diffuser device on the wall.

10. The oil diffuser device of claim 9, wherein the plug-in component comprises a fan to direct air and the heated CBD oil vapors from the body component out through the plurality of ventilation holes.

11. The oil diffuser device of claim 10, wherein the plug-in component comprises a heat source that heats the CBD oil.

12. The oil diffuser device of claim 11, wherein the heat source includes a wire wound resistor and a thermostat which are all embedded into the plug-in component.

13. The oil diffuser device of claim 12, wherein the heated CBD oil vapors are released when the CBD oil is heated and the heated CBD oil vapors are directed upwards by airflow from the fan and then passed through the plurality of ventilated holes of the body component out into the air.

14. An oil diffuser device that provides a user with a plug-in device for diffusing CBD oil into the air, the oil diffuser device comprising:
a body component comprising a bottom surface and a curved sidewall configured in a cylindrical shape defining a cavity, with an open top that allows access to the cavity; and
a plug-in component comprising a power plug component for securing to an electrical outlet;
wherein the body component is configured to retain an amount of CBD oil;
wherein the open top comprises a plurality of external threads which mate with a plurality of internal threads on the plug-in component to secure the body component to the plug-in component;
wherein the body component comprises a plurality of ventilation holes positioned near the open top;
wherein the plug-in component comprises a fan to direct air and heated CBD oil vapors from the body component out through the plurality of ventilation holes;
wherein the plug-in component comprises a heat source that heats the CBD oil;
wherein the plug-in component is inserted into an electrical outlet to heat the CBD oil and release the heated CBD oil vapors into the air; and
further wherein the heated CBD oil vapors are released when the CBD oil is heated and the heated CBD oil vapors are directed upwards by airflow from the fan and then passed through the plurality of ventilated holes of the body component out into the air.

15. The oil diffuser device of claim 14, wherein the plug-in component comprises a light source electrically coupled to the power plug component.

16. The oil diffuser device of claim 15, wherein the light source includes at least one LED.

17. The oil diffuser device of claim 16, wherein the plug-in component comprises a light control button that controls color and brightness of the light source and power.

18. The oil diffuser device of claim 14, wherein the plug-in component comprises a faceplate to cover and hide the body component for a more aesthetically pleasing appearance.

19. The oil diffuser device of claim 14 further comprising a plurality of indicia.

20. A method of diffusing CBD oil into the air, the method comprising the following steps:
providing an oil diffuser device comprising a body component with ventilation holes and a plug-in component;
inserting CBD oil into the body component;
securing the body component to the plug-in component;
inserting the plug-in component into an electrical outlet;
heating the CBD oil with a heat source in the plug-in component; and
dispersing heated CBD oil vapors through the plurality of ventilation holes via a fan.
